(19) United States
(12) Patent Application Publication
(10) Pub. No.: US 2024/0428947 A1
(43) Pub. Date: Dec. 26, 2024

(54) Method And System For Predicting A Clinical Outcome Of Metabolic Dysfunction-Associated Steatotic Liver Disease

(71) Applicant: Histoindex Pte Ltd, Singapore (SG)

(72) Inventor: Chi Shang TAI, Singapore (SG)

(73) Assignee: Histoindex Pte Ltd, Singapore (SG)

(21) Appl. No.: 18/748,820

(22) Filed: Jun. 20, 2024

(30) Foreign Application Priority Data
Jun. 21, 2023 (SG) 10202301763Y

(51) Int. Cl. G16H 50/30; G16B 15/00; G16H 50/20

(52) U.S. Cl. CPC G16H 50/30; G16B 15/00; G16H 50/20

(57) ABSTRACT

A method for predicting a clinical outcome of metabolic dysfunction-associated steatotic liver disease (MASLD) includes imaging a liver biopsy sample having MASLD to obtain image data. An initial set of fibrosis parameters is generated from the image data. An index indicative of the clinical outcome is calculated based on the initial set of fibrosis parameters.

### CROSS-REFERENCE TO RELATED APPLICATION

This application claims priority to Singapore Patent Application No. 10202301763Y, the contents of which are incorporated herein by reference in their entirety.

### TECHNICAL FIELD

The present disclosure relates broadly, but not exclusively, to methods and systems for predicting a clinical outcome of metabolic dysfunction-associated steatotic liver disease.

### BACKGROUND

Metabolic dysfunction-associated steatotic liver disease (MASLD), previously termed non-alcoholic fatty liver disease (NAFLD), is an increasing and under-appreciated global public health challenge that affects more than one in four adults. Risk factors include obesity, type 2 diabetes, hypertension, and dyslipidemia. MASLD is associated with increased morbidity and mortality from cardiovascular disease, chronic kidney disease, and many cancers. The histopathological disease spectrum includes fatty liver in the absence of inflammation (simple steatosis) and metabolic dysfunction-associated steatohepatitis (MASH), formerly termed non-alcoholic steatohepatitis (NASH), the inflammatory progressive form of MASLD that can lead to liver fibrosis, cirrhosis, and hepatocellular carcinoma (HCC). Critically, not all people with MASLD develop adverse liver-related events or die, but there is a lack of prognostic biomarkers to allow the implementation of personalised approaches to disease management.

Further, there are no licensed treatments for MASLD or MASH, and many candidate drugs have failed in late-phase clinical trials. Regulatory authorities currently mandate ordinal scoring by a pathologist to quantify both disease stage (NASH-CRN fibrosis score) and disease activity (NAFLD Activity Score (NAS)) in clinical trials, and efficacy of candidate treatments is judged by change in those scores.

Fibrosis stage in MASLD is strongly predictive of clinical outcomes such that improvement in ordinal score is considered an acceptable surrogate for clinically meaningful treatment. However, reliance on subjective assessment introduces inherent intra- and inter-rater variation. Computational methods have been developed to assign disease stage to reduce such variation, although the impact of variation in staining characteristics that can be easily ignored by a human observer is challenging to overcome. Further, the wisdom of forcing a bidirectional dynamic process into discrete ordinal categories is questionable, whether computationally or manually assigned.

It may be desirable to provide methods and devices that can predict clinical outcomes of MASLD that can address at least some of the above problems.

### SUMMARY

An aspect of the present disclosure provides a method for predicting a clinical outcome of metabolic dysfunction-associated steatotic liver disease (MASLD), comprising imaging a liver biopsy sample having MASLD to obtain image data; generating an initial set of fibrosis parameters from the image data; and calculating an index indicative of the clinical outcome based on the initial set of fibrosis parameters.

Calculating the index may comprise selecting a reduced set of fibrosis parameters from the initial set of fibrosis parameters using sequential feature selection; and calculating the index from the reduced set of fibrosis parameters using linear regression.

The index comprises an all-cause mortality index, and the reduced set of fibrosis parameters may comprise selected first parameters. The selected first parameters may comprise two portal features, one peri-portal features and two zone 2 features.

The index may comprise a hepatic decompensation index, and the reduced set of fibrosis parameters may comprise selected second parameters. The selected second parameters may comprise one portal feature and four periportal features.

The index comprises a hepatocellular carcinoma index, and the reduced set of fibrosis parameters may comprise selected third parameters.

The method may further comprise classifying a risk of the clinical outcome based on the index.

Imaging the liver biopsy sample may comprise a second harmonic generation/two-photon excitation fluorescence (SHG/TPEF) microscopy.

Another aspect of the present disclosure provides a system for predicting a clinical outcome of metabolic dysfunction-associated steatotic liver disease (MASLD), the system comprising a processor and a computer-readable memory coupled to the processor and having instructions stored thereon that are executable by the processor to receive image data of a liver biopsy sample, the liver biopsy sample having MASLD, generate an initial set of fibrosis parameters from the image data, and calculate an index indicative of the clinical outcome based on the initial set of fibrosis parameters.

The instructions to calculate the index may comprise instructions that are executable by the processor to select a reduced set of fibrosis parameters from the initial set of fibrosis parameters using sequential feature selection, and calculate the index from the reduced set of fibrosis parameters using linear regression.

The index comprises an all-cause mortality index, and the reduced set of fibrosis parameters may comprise selected first parameters. The selected first parameters may comprise two portal features, one peri-portal features and two zone 2 features.

The index may comprise a hepatic decompensation index, and the reduced set of fibrosis parameters may comprise selected second parameters. The selected second parameters may comprise one portal feature and four periportal features.

The index comprises a hepatocellular carcinoma index, and the reduced set of fibrosis parameters may comprise selected third parameters.

The instructions that are executable by the processor may further comprise instructions to classify a risk of the clinical outcome based on the index.

The image data may comprise data from a second harmonic generation/two-photon excitation fluorescence (SHG/TPEF) microscope.

### BRIEF DESCRIPTION OF THE DRAWINGS

Embodiments of the disclosure will be better understood and readily apparent to one of ordinary skill in the art from the following written description, by way of example only, and in conjunction with the drawings, in which:

FIG. 1 shows a flow chart illustrating a method for predicting a clinical outcome of metabolic dysfunction-associated steatotic liver disease according to an example embodiment.

FIG. 2 shows graphs comparing probability of a clinical outcome when the index is greater than a threshold against when the index less than the threshold, for mortality, decompensation and HCC.

FIG. 3 shows predictive performance of the all-cause mortality index in Example 1 compared to NASH-CRN and qFibrosis.

FIG. 4 shows predictive performance of the decompensation index in Example 1 compared to NASH-CRN and qFibrosis.

FIG. 5 shows predictive performance of the HCC index in Example 1 compared to NASH-CRN and qFibrosis.

FIG. 6 shows boxplots of individual Mortality Index parameter values in Example 2 across the NASH-CRN fibrosis stage spectrum.

FIG. 7 shows predictive performance of the Mortality Index in Example 2 compared to NASH-CRN and qFibrosis.

FIG. 8 shows boxplots of individual Decompensation parameter values across the NASH-CRN fibrosis stage spectrum.

FIG. 9 shows predictive performance of the Decompensation Index in Example 2 compared to NASH-CRN and qFibrosis.

FIG. 10 shows a schematic diagram of a computer system for implementing the method of FIG. 1.

Skilled artisans will appreciate that elements in the figures are illustrated for simplicity and clarity and have not necessarily been depicted to scale. For example, the dimensions of some of the elements in the illustrations, block diagrams or flowcharts may be exaggerated in respect to other elements to help to improve understanding of the present embodiments.

### DETAILED DESCRIPTION

The present disclosure seeks to use stain-free imaging to generate tools predictive of patient outcomes using architectural features unapparent to human observers, and to establish individual indices for risk of all-cause mortality, hepatic decompensation and hepatocellular carcinoma (HCC) from key fibrotic architectural features identified using stain-free imaging.

Embodiments will be described, by way of example only, with reference to the drawings. Like reference numerals and characters in the drawings refer to like elements or equivalents.

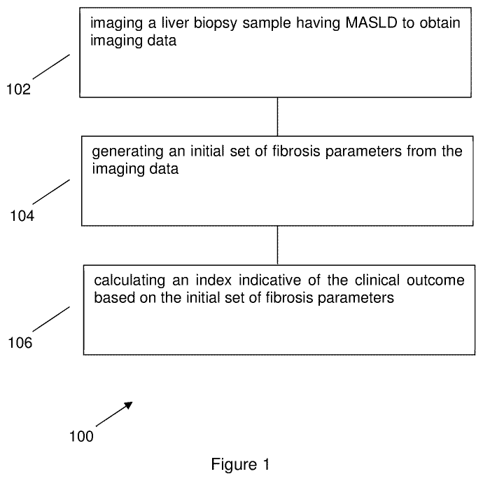

FIG. 1 shows a flow chart 100 illustrating a method for predicting a clinical outcome of metabolic dysfunction-associated steatotic liver disease (MASLD) according to an example embodiment. At step 102, a liver biopsy sample having MASLD is imaged to obtain image data. At step 104, an initial set of fibrosis parameters is generated from the image data. At step 106, an index indicative of the clinical outcome is calculated based on the initial set of fibrosis parameters.

As further described below, the imaging in step 102 may be performed using a second harmonic generation/two-photon excitation fluorescence (SHG/TPEF) microscopy. Step 106 may include selecting a reduced set of fibrosis parameters from the initial set of fibrosis parameters using sequential feature selection and calculating the index from the reduced set of fibrosis parameters using linear regression. The index may be an all-cause mortality index, a hepatic decompensation index, or a hepatocellular carcinoma index. The features in the reduced set of fibrosis features may be adapted accordingly based on the index to be calculated.

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “scanning”, “calculating”, “determining”, “applying”, “extracting”, “generating”, “initializing”, “outputting”, or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a computer or other device selectively activated or reconfigured by a computer program stored in the computer. The algorithms and displays presented herein are not inherently related to any particular computer or other apparatus. Various machines may be used with programs in accordance with the teachings herein. Alternatively, the construction of more specialized apparatus to perform the required method steps may be appropriate. The structure of a conventional computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the scope of the disclosure.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a computer. The computer readable medium may also include a hard-wired medium such as exemplified in the Internet system, or wireless medium such as exemplified in the GSM, GPRS, 3G, 4G or 5G mobile telephone systems, as well as other wireless systems such as Bluetooth, ZigBee, Wi-Fi. The computer program when loaded and executed on such a computer effectively results in an apparatus that implements the steps of the preferred method.

The present disclosure may also be implemented as hardware elements. More particularly, in the hardware sense, an element is a functional hardware unit designed for use with other components or elements. For example, an element may be implemented using discrete electronic components, or it can form a portion of an entire electronic circuit such as an Application Specific Integrated Circuit (ASIC) or Field Programmable Gate Array (FPGA). Numerous other possibilities exist. Those skilled in the art will appreciate that the system can also be implemented as a combination of hardware and software elements.

According to various embodiments, a “circuit” may be understood as any kind of a logic implementing entity, which may be special purpose circuitry or a processor executing software stored in a memory, firmware, or any combination thereof. Thus, in an embodiment, a “circuit” may be a hard-wired logic circuit or a programmable logic circuit such as a programmable processor, e.g. a microprocessor (e.g. a Complex Instruction Set Computer (CISC) processor or a Reduced Instruction Set Computer (RISC) processor). A “circuit” may also be a processor executing software, e.g. any kind of computer program, e.g. a computer program using a virtual machine code such as e.g. Java. Any other kind of implementation of the respective functions which may be described in more detail herein may also be understood as a “circuit” in accordance with an alternative embodiment.

### Example 1

In this example, unstained sections from a training set of 220 biopsies from SteatoSITE, a resource containing integrated clinical and pathological data from 940 cases across the NAFLD spectrum, were imaged using second harmonic generation/two-photon excitation fluorescence microscopy. Feature selection was then performed to reduce the dimensionality of data by selecting only a subset of collagen features. For example, using sequential feature selection, 10, 10 and 5 parameters were chosen out of 184 fibrosis parameters and a linear regression method was used to construct individual indices for risk of all-cause mortality, hepatic decompensation and hepatocellular carcinoma (HCC), respectively. In the linear regression model used, the criterion was the residual sum of squares, and the search algorithm was sequential forward selection.

In other words, a total of 220 samples were used to find the most significant collagen features related with all-cause mortality, hepatic decompensation and hepatocellular carcinoma (HCC). Secondly, a model was trained to predict risk in patients using an “individual index”, which was constructed from the previously mentioned 220 cases with multivariable linear regression method. To validate the prediction model, leave-one-out cross-validation method was used.

Time-to-event analysis was performed using the Kaplan-Meier method, with death as a competing risk for decompensation and HCC, and distributions compared using the log-rank test. A Cox proportional hazards model was used to estimate hazard ratios (HRs).

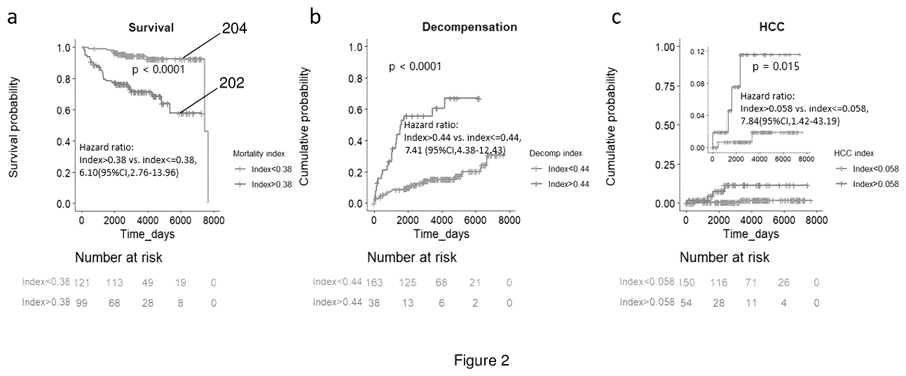

FIG. 2 shows probability graphs for all-cause mortality, hepatic decompensation and HCC, respectively. From FIG. 2, the survival probability drops significantly if the index is greater than a threshold (which is 0.38 in this instance), as shown by line 202 being significantly lower than line 204 throughout the whole period). In other words, the risk of mortality is significantly higher if the index is higher than the threshold. Similarly, the risk of decompensation increases substantially if the index is higher than a threshold of 0.44, while the risk of HCC also increases substantially if the index is higher than a threshold of 0.058. These graphs show that it is possible to classify a risk of a clinical outcome by setting an appropriate threshold of the corresponding index.

The predictive power of the risk indices was compared against existing approaches, namely, (1) the assigned NASH-CRN fibrosis stage (F0/1/2 v F3/4) and (2) the stain-free imaging derived qFibrosis stage (qF0/1/2 v qF3/4). qFibrosis is a machine learning-based algorithm with second harmonic generation/two-photon excitation fluorescence (SHG/TPEF) imaging that provides a visual mapping of collagen burden/distribution & permits measurement of quantifiable collagen fibrillar properties, e.g. length, width and area of fibers according to their distribution in the histopathological regions. A previous longitudinal study had demonstrated the potential prognostic significance of qFibrosis parameters where subjects with high qFibrosis parameters had increased incidence of liver-related events.

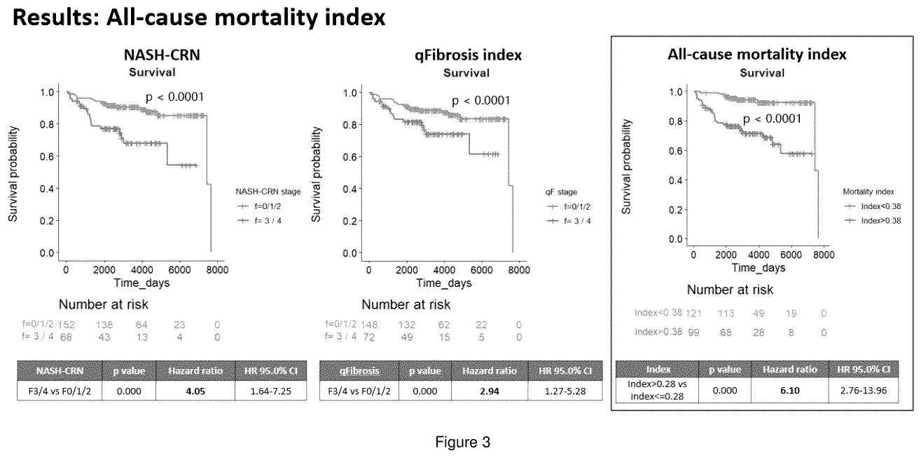

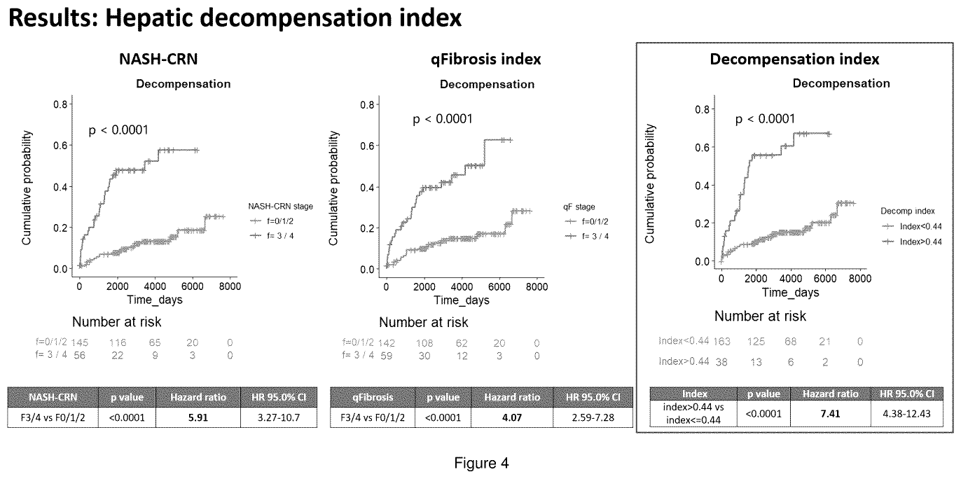

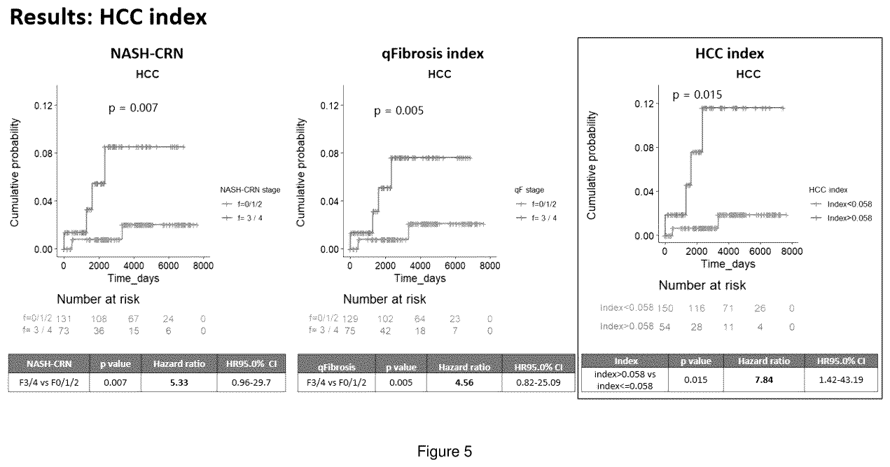

FIGS. 3-5 show the performance of the all-cause mortality, hepatic decompensation and HCC indices, respectively, when compared against NASH-CRN and qFibrosis. It can be observed that the “All-cause Mortality Index” had greater predictive power for all-cause mortality (index>0.38 vs. ≤0.38, HR 6.10, 95% confidence intervals (CI) 2.76-13.96, p<0.0001) than either NASH-CRN or qFibrosis stage. The “Decompensation Index” had greater predictive power for decompensation events (index>0.44 vs. ≤0.44, HR 7.41, 95% CI 4.38-12.43, p<0.0001) than either NASH-CRN stage or qFibrosis stage. Finally, the “HCC Index” had greater predictive power for HCC development (index>0.058 vs. ≤0.058, HR 7.84, 95% CI 1.42-43.19, p=0.015) than either NASH-CRN stage or qFibrosis stage.

### Example 2

In this example, 300 needle liver biopsy cases from SteatoSITE were used. Decompensation events in the clinical data extract were those defined by a combination of World Health Organisation International Classification of Diseases version 9 and 10 (ICD-9/10) codes and United Kingdom National Health Service OPCS Classification of Interventions and Procedures version 4 (OPCS-4) codes identifying activity relating to cirrhosis-related hospital admissions activity. Decompensation and HCC event analysis was only undertaken on biopsy cases where the first decompensation or HCC-related coding was present in the clinical data extract after the recorded biopsy date, and analysis was undertaken using death as a competing risk.

A single unstained, formalin-fixed section from each of the 300 liver biopsies from SteatoSITE were examined using SHG/TPEF microscopy with computer-assisted analyses. The liver sections were de-paraffinized, followed by tissue scanning on Genesis®200 (a fully automated, stain-free TPEF imaging microscope). Samples were laser-excited at 780 nm, SHG signals were recorded at 390 nm, and TPEF signals were recorded at 550 nm. Images were acquired at 20× magnification with a 512×512 pixels resolution; each image tile had a dimension of 200×200 mm. Multiple adjacent image tiles were captured to encompass the whole tissue areas in each slide. 184 separate continuous outputs each relating to distinct collagen architectural and topographical features were derived. The total amount and distribution of liver fibrosis were quantitatively assessed by calculation of the single qFibrosis metric, an overall output derived from the composite variables. qFibrosis calculation is based on normalized collagen parameters expressed as the number of units per mm².

Cases were randomly split into training (n=200) and validation (n=100) groups. Sequential feature selection was used to reduce the number of parameters and create the sparsest possible models. Five and five of 184 fibrosis parameters were chosen, and a linear regression method was used to construct individual indices for risk of all-cause mortality and hepatic decompensation, respectively. A threshold value dividing cases into high and low risk was calculated by Youden's index.

In other words, to construct individual indices for risk of all-cause mortality and hepatic decompensation, a prediction model was developed based on the quantified collagen features.

Firstly, feature selection was performed to reduce the dimensionality of data by selecting only a subset of collagen features. In the example embodiment, sequential feature selection was used. In the procedure of sequential feature selection, a linear regression model was used whereby the criterion was the residual sum of squares and the search algorithm was sequential forward selection.

In a total of 300 cases, 200 cases in the training group were used to find the most significant collagen features related with all-cause mortality and hepatic decompensation. Next, a model was trained to predict risk in patients using an “individual index”, which was constructed from the previously mentioned 200 cases with multivariable linear regression method. Next, the validation group of 100 cases was used to validate the model.

Time-to-event analysis was performed using the Kaplan-Meier method, with death as a competing risk for decompensation, and distributions were compared using the log-rank test. A Cox proportional hazards model was used to estimate hazard ratios after determining that the assumptions necessary were met through calculation of Schoenfield residuals and outlier detection using deviance residuals. The predictive power of the risk indices was compared with the ordinal NASH-Clinical Research Network (CRN) fibrosis stage (F0/1/2 v F3/4) and the stain-free imaging-derived qFibrosis stage (qF0/1/2 v qF3/4). A p-value of <0.05 was considered statistically significant.

Tables 1 show the hazard ratios and 95% CI for NASH-CRN, qFibrosis, and all-cause mortality index, while Table 2 shows the hazard ratios and 95% CI for NASH-CRN, qFibrosis, and decompensation index, for both the training and validation cohorts. The all-cause mortality and decompensation indices provided by the present method have greater predictive power than both NASH-CRN and qFibrosis, as shown by the relatively higher hazard ratios for these indices in Tables 1 and 2.

TABLE 1

| All-cause | | | Hazard | HR 95.0% |
|---|---|---|---|---|
| mortality | Method | p value | ratio | CI |
| Training | NASH-CRN | 0.001 | 4.83 | 2.39-13.22 |
| | qFibrosis stage | 0.005 | 4.33 | 1.74-14.59 |
| | All-cause mortality index | 0.002 | 5.88 | 1.67-19.30 |
| Validation | NASH-CRN | 0.0051 | 3.77 | 1.39-10.20 |
| | qFibrosis stage | 0.0038 | 3.91 | 1.45-10.59 |
| | All-cause mortality index | 0.016 | 5.19 | 1.17-23.00 |

TABLE 2

| | | | Hazard | HR 95.0% |
|---|---|---|---|---|
| Decompensation | Method | p value | ratio | CI |
| Training | NASH-CRN | 0.003 | 4.56 | 1.58-14.22 |
| | qFibrosis stage | 0.0002 | 4.24 | 1.00-13.99 |
| | Decompensation index | 0.0003 | 7.34 | 2.67-23.11 |
| Validation | NASH-CRN | 0.0018 | 3.43 | 1.51-7.81 |
| | qFibrosis stage | 0.0001 | 3.70 | 1.61-8.49 |
| | Decompensation index | <0.000 | 5.26 | 2.27-12.17 |

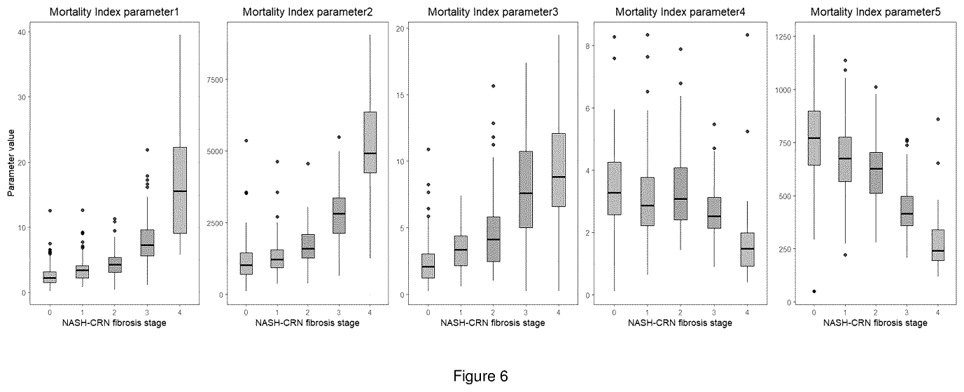

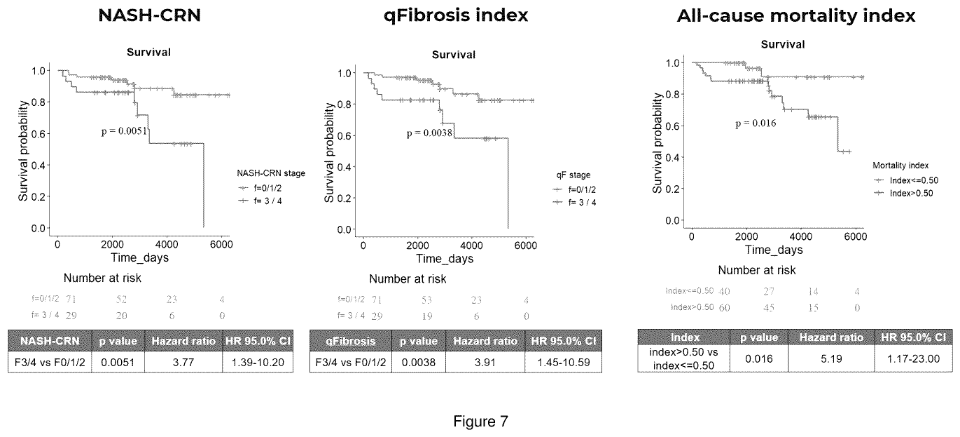

In the validation cohort, the all-cause mortality index comprising 5 parameters had greater predictive power based on a larger hazard ratio (HR) (Mortality Index≤0.50 v>0.50, HR 5.19, confidence intervals (CI) 1.17-23, p=0.016) than either pathologist-assigned NASH-CRN (F0/1/2 v F3/4, HR 3.77, 95% CI 1.39-10.20, p=0.0051) or qFibrosis stage (qF0/1/2 v qF3/4, HR 3.91, 95% CI 1.45-10.59, p=0.0038). Two of the five parameters were portal features, one was a peri-portal feature, and two were zone 2 features. FIG. 6 shows boxplots of individual Mortality Index parameter values across the NASH-CRN fibrosis stage spectrum. FIG. 7 shows Kaplan-Meier time-to-event analysis in n=100 validation cohort with log-rank test p-value for all-cause mortality with high/low risk dichotomisation based on Mortality Index (index≤0.50 vs. >0.50 vs, p=0.016), NASH-CRN fibrosis stage (stage F0,1,2 vs. F3,4, p=0.0038) and qFibrosis stage (qFibrosis stage qF0,1,2 vs. qF3,4. p=0.0051).

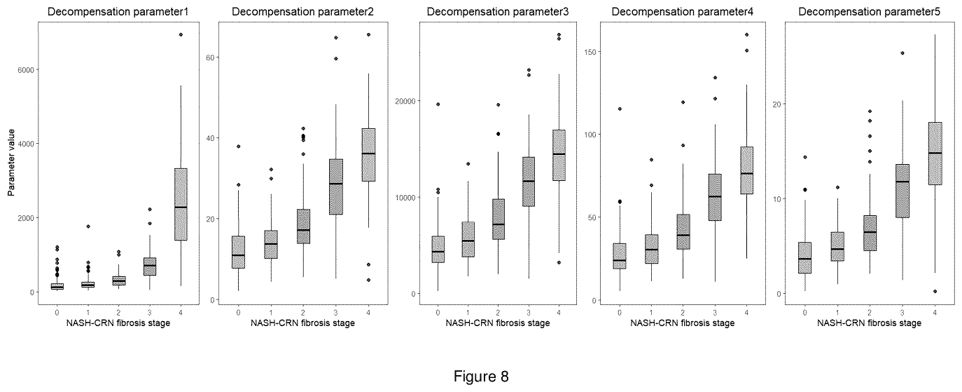

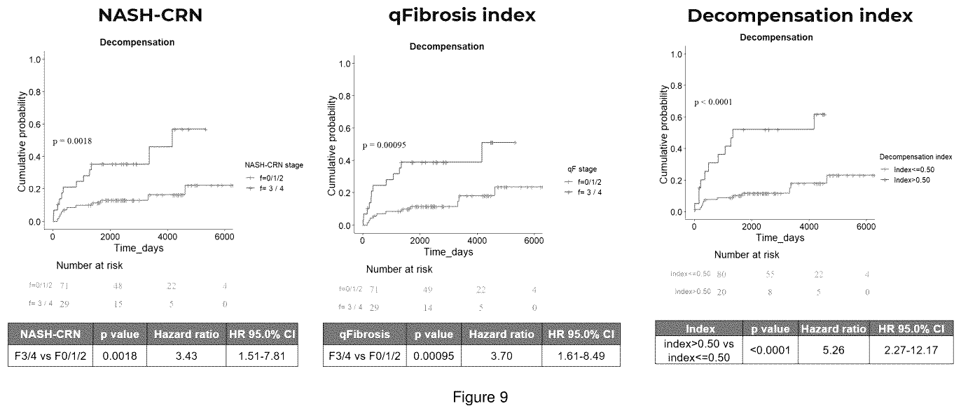

The hepatic decompensation index, also comprising 5 parameters, had greater predictive power based on a larger HR (Hepatic Decompensation Index≤0.50 v>0.50, HR 5.26, 95% CI 2.27-12.17, p<0.0001) than either pathologist-assigned NASH-CRN (F0/1/2 v F3/4, HR 3.43, 95% CI 1.51-7.81, p=0.0018) or qFibrosis stage (qF0/1/2 v qF3/4, HR 3.70, 95% CI 1.61-8.49, p=0.00095). One of the five parameters was a portal feature, and four were periportal features. FIG. 8 shows boxplots of individual Hepatic Decompensation Index parameter values across the NASH-CRN fibrosis stage spectrum. FIG. 9 shows Kaplan-Meier time-to-event analysis in n=100 validation cohort with log-rank test p-value for hepatic decompensation (with death as a competing risk) with high/low risk dichotomisation based on Hepatic Decompensation Index (index≤0.50 vs. >0.50, p<0.0001), NASH-CRN fibrosis stage (stage F0,1,2 vs. F3,4, p=0.0018) and qFibrosis stage (qFibrosis stage qF0,1,2 vs. qF3,4. p=0.00095).

The above examples show that the method of the present disclosure can directly predict hard endpoints in patients with NAFLD and does not rely on ordinal fibrosis scores as a surrogate. The indices provided by the present method have greater predictive value than pathologist-assigned NASH-CRN fibrosis stage or computationally-assigned qFibrosis stage. These indices may provide direct tissue-to-outcome predictions that aid clinical decision-making, offer more nuanced participant stratification and meaningful endpoints in clinical trials.

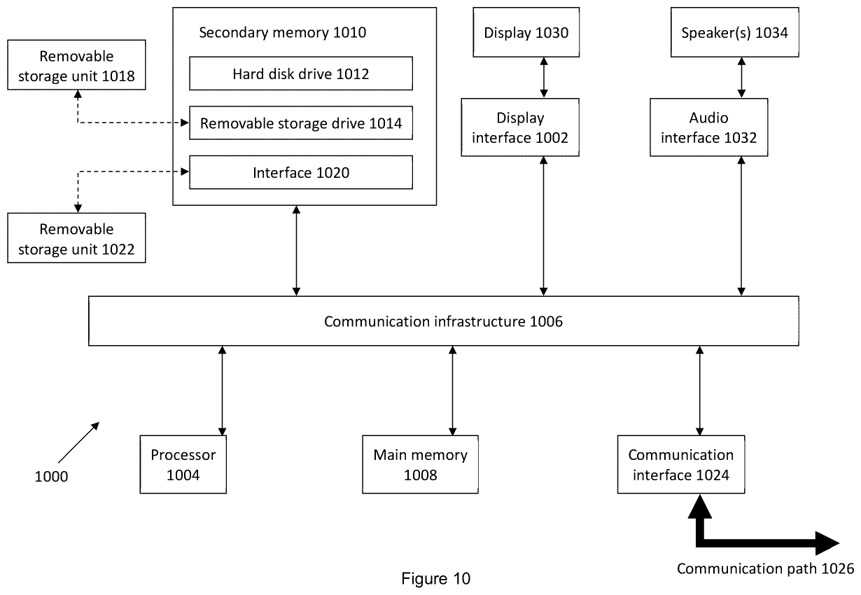

FIG. 10 depicts an exemplary computing device 1000, hereinafter interchangeably referred to as a computer system 1000, where one or more such computing devices 1000 may be used to implement the method as described. The following description of the computing device 1000 is provided by way of example only and is not intended to be limiting.

As shown in FIG. 10, the example computing device 1000 includes a processor 1004 for executing software routines. Although a single processor is shown for the sake of clarity, the computing device 1000 may also include a multi-processor system. The processor 1004 is connected to a communication infrastructure 1006 for communication with other components of the computing device 1000. The communication infrastructure 1006 may include, for example, a communications bus, cross-bar, or network.

The computing device 1000 further includes a main memory 1008, such as a random access memory (RAM), and a secondary memory 1010. The secondary memory 1010 may include, for example, a hard disk drive 1012 and/or a removable storage drive 1014, which may include a floppy disk drive, a magnetic tape drive, an optical disk drive, or the like. The removable storage drive 1014 reads from and/or writes to a removable storage unit 1018 in a well-known manner. The removable storage unit 1018 may include a floppy disk, magnetic tape, optical disk, or the like, which is read by and written to by removable storage drive 1014. As will be appreciated by persons skilled in the relevant art(s), the removable storage unit 1018 includes a computer readable storage medium having stored therein computer executable program code instructions and/or data.

In an alternative implementation, the secondary memory 1010 may additionally or alternatively include other similar means for allowing computer programs or other instructions to be loaded into the computing device 1000. Such means can include, for example, a removable storage unit 1022 and an interface 1020. Examples of a removable storage unit 1022 and interface 1020 include a program cartridge and cartridge interface (such as that found in video game console devices), a removable memory chip (such as an EPROM or PROM) and associated socket, and other removable storage units 1022 and interfaces 1020 which allow software and data to be transferred from the removable storage unit 1022 to the computer system 1000.

The computing device 1000 also includes at least one communication interface 1024. The communication interface 1024 allows software and data to be transferred between computing device 1000 and external devices via a communication path 1026. In various embodiments of the disclosure, the communication interface 1024 permits data to be transferred between the computing device 1000 and a data communication network, such as a public data or private data communication network. The communication interface 1024 may be used to exchange data between different computing devices 1000 which such computing devices 1000 form part an interconnected computer network. Examples of a communication interface 1024 can include a modem, a network interface (such as an Ethernet card), a communication port, an antenna with associated circuitry and the like. The communication interface 1024 may be wired or may be wireless. Software and data transferred via the communication interface 1024 are in the form of signals which can be electronic, electromagnetic, optical or other signals capable of being received by communication interface 1024. These signals are provided to the communication interface via the communication path 1026.

As shown in FIG. 10, the computing device 1000 further includes a display interface 1002 which performs operations for rendering images to an associated display 1030 and an audio interface 1032 for performing operations for playing audio content via associated speaker(s) 1034.

As used herein, the term “computer program product” may refer, in part, to removable storage unit 1018, removable storage unit 1022, a hard disk installed in hard disk drive 1012, or a carrier wave carrying software over communication path 1026 (wireless link or cable) to communication interface 1024. Computer readable storage media refers to any non-transitory tangible storage medium that provides recorded instructions and/or data to the computing device 1000 for execution and/or processing. Examples of such storage media include floppy disks, magnetic tape, CD-ROM, DVD, Blu-ray™ Disc, a hard disk drive, a ROM or integrated circuit, USB memory, a magneto-optical disk, or a computer readable card such as a PCMCIA card and the like, whether or not such devices are internal or external of the computing device 1000. Examples of transitory or non-tangible computer readable transmission media that may also participate in the provision of software, application programs, instructions and/or data to the computing device 1000 include radio or infra-red transmission channels as well as a network connection to another computer or networked device, and the Internet or Intranets including e-mail transmissions and information recorded on Websites and the like.

The computer programs (also called computer program code) are stored in main memory 1008 and/or secondary memory 1010. Computer programs can also be received via the communication interface 1024. Such computer programs, when executed, enable the computing device 1000 to perform one or more features of embodiments discussed herein. In various embodiments, the computer programs, when executed, enable the processor 1004 to perform features of the above-described embodiments. Accordingly, such computer programs represent controllers of the computer system 1000.

Software may be stored in a computer program product and loaded into the computing device 1000 using the removable storage drive 1014, the hard disk drive 1012, or the interface 1020. Alternatively, the computer program product may be downloaded to the computer system 1000 over the communications path 1026. The software, when executed by the processor 1004, causes the computing device 1000 to perform functions of embodiments described herein.

It is to be understood that the embodiment of FIG. 10 is presented merely by way of example. Therefore, in some embodiments one or more features of the computing device 1000 may be omitted. Also, in some embodiments, one or more features of the computing device 1000 may be combined together. Additionally, in some embodiments, one or more features of the computing device 1000 may be split into one or more component parts.

It will be appreciated that the elements illustrated in FIG. 10 function to provide means for performing the various functions and operations of the servers as described in the above embodiments.

In an implementation, a server may be generally described as a physical device comprising at least one processor and at least one memory including computer program code. The at least one memory and the computer program code are configured to, with the at least one processor, cause the physical device to perform the requisite operations.

It will be appreciated by a person skilled in the art that numerous variations and/or modifications may be made to the present disclosure as shown in the specific embodiments without departing from the scope of the disclosure as broadly described. The present embodiments are, therefore, to be considered in all respects to be illustrative and not restrictive.

1. A method for predicting a clinical outcome of metabolic dysfunction-associated steatotic liver disease (MASLD), comprising:
imaging a liver biopsy sample having MASLD to obtain image data;
generating an initial set of fibrosis parameters from the image data; and
calculating an index indicative of the clinical outcome based on the initial set of fibrosis parameters.

2. The method as claimed in claim 1, wherein calculating the index comprises:
selecting a reduced set of fibrosis parameters from the initial set of fibrosis parameters using sequential feature selection; and
calculating the index from the reduced set of fibrosis parameters using linear regression.

3. The method as claimed in claim 1, wherein the index comprises an all-cause mortality index, and wherein the reduced set of fibrosis parameters comprises selected first parameters.

4. The method as claimed in claim 3, wherein the selected first parameters comprise two portal features, one peri-portal features and two zone 2 features.

5. The method as claimed in claim 1, wherein the index comprises a hepatic decompensation index, and wherein the reduced set of fibrosis parameters comprises selected second parameters.

6. The method as claimed in claim 5, wherein the selected second parameters comprise one portal feature and four periportal features.

7. The method as claimed in claim 1, wherein the index comprises a hepatocellular carcinoma index, and wherein the reduced set of fibrosis parameters comprises selected third parameters.

8. The method as claimed in claim 1, further comprising classifying a risk of the clinical outcome based on the index.

9. The method as claimed in claim 1, wherein imaging the liver biopsy sample comprises a second harmonic generation/two-photon excitation fluorescence (SHG/TPEF) microscopy.

10. A system for predicting a clinical outcome of metabolic dysfunction-associated steatotic liver disease (MASLD), the system comprising:
a processor;
a computer-readable memory coupled to the processor and having instructions stored thereon that are executable by the processor to:
receive image data of a liver biopsy sample, the liver biopsy sample having MASLD;
generate an initial set of fibrosis parameters from the image data; and
calculate an index indicative of the clinical outcome based on the initial set of fibrosis parameters.

11. The system as claimed in claim 10, wherein the instructions to calculate the index comprise instructions that are executable by the processor to:
select a reduced set of fibrosis parameters from the initial set of fibrosis parameters using sequential feature selection; and
calculate the index from the reduced set of fibrosis parameters using linear regression.

12. The system as claimed in claim 10, wherein the index comprises an all-cause mortality index, and wherein the reduced set of fibrosis parameters comprises selected first parameters.

13. The system as claimed in claim 12, wherein the selected first parameters comprise two portal features, one peri-portal features and two zone 2 features.

14. The system as claimed in claim 10, wherein the index comprises a hepatic decompensation index, and wherein the reduced set of fibrosis parameters comprises selected second parameters.

15. The system as claimed in claim 14, wherein the selected second parameters comprise one portal feature and four periportal features.

16. The system as claimed in claim 10, wherein the index comprises a hepatocellular carcinoma index, and wherein the reduced set of fibrosis parameters comprises selected third parameters.

17. The system as claimed in claim 10, wherein the instructions that are executable by the processor further comprise instructions to classify a risk of the clinical outcome based on the index.

18. The system as claimed in claim 10, wherein the image data comprises data from a second harmonic generation/two-photon excitation fluorescence (SHG/TPEF) microscope.
